(19) United States
(12) Patent Application Publication
(10) Pub. No.: US 2017/0027780 A1
(43) Pub. Date: Feb. 2, 2017

(54) McCoy Cast Covers

(71) Applicants: Stuart D. McCoy, Peru, IN (US); Joan M. McCoy, Peru, IN (US)

(72) Inventors: Stuart D. McCoy, Peru, IN (US); Joan M. McCoy, Peru, IN (US)

(21) Appl. No.: 14/814,481

(22) Filed: Jul. 30, 2015

(51) Int. Cl. A61F 15/00; D05B 23/00; A61F 13/00

(52) U.S. Cl. CPC A61F 15/004; A61F 13/00987; A61F 13/00059; A61F 13/00085; D05B 23/006

(57) ABSTRACT

Disclosed herein is a disposable, yet re-usable and affordable design that will offer protection for the wearer from harmful or damaging fluids, moisture or other contaminants. Further the effective covering designed for comfort and single-handed application to allow the wearer adequate coverage for normal daily routines, such as bathing, and more independence to perform these activities, making recovery time simpler and easier. Also the invention includes the use of a water-resistant plastic material preferably clear, colored or printed, formed into a protective cover for the limbs in or other body areas and gathered at the openings with elastic band sealed to the edges, and in some items, with a second band sealed into a cuff in the interior of the device, a distance parallel to the band on the end. The design will avoid circulatory problems of over-tightening and the problems of the other devices to secure a reasonably leak-proof opening.

### CROSS REFERENCE TO RELATED APPLICATIONS

Not applicable.

### FEDERALLY SPONSORED RESEARCH AND DEVELOPMENT

Not applicable.

### MICROFICHE

Not applicable

### BACKGROUND OF THE INVENTION

(1) Field of the Invention

The invention of these devices relates to the protection of various body parts from damaging moisture or other contaminants, during bathing procedures, normal daily activities, or other uses when the wearer wishes an area to be kept clean, dry or protected. More particularly, related with disposable, but re-usable devices elasticized at the openings, are simply designed for single-handed application and contain no additionally attached or unattached straps, ties or closures.

(2) Background

Casts and bandages are placed on extremities for lengthy periods of time and can be a source of considerable discomfort. Since casts or bandages should be kept dry while they are on the extremity, bathing, showering, and the like, are difficult. If water soaks into the cast or bandage or penetrates beneath it, the patient will experience discomfort in the form of itching, infection and odor emissions.

Covers for casts and bandages constructed of plastic materials are well known to the art. For example, few devices disclose a sack-like, water-impervious covering which includes an upper lip with perforations which extend parallel to the opening. An adhesive strip extends over the entire width of the lip from one end to the other. The adhesive trip is covered by a removable sheet to protect the adhesive prior to use. In use, the perforation is torn to separate the lip from the sack and form a tie member. The adhesive protection sheet is removed and the extremity is inserted into the sack-like covering. A securing portion is then is adhered to the surface of the extremity and the opening is gathered around the extremity so that the tie member can be used to encircle the gathering to cover the extremity and prevent the entry of water. Such devices, however, are not reusable since once the adhesive strip is exposed by removing the sheet and the cover is gathered to tighten it around the extremity, the only way the cover can be removed is to destroy it. Since casts are kept on for generally four to six weeks, a significant number of these covers must be used if the wearer wants to bathe or shower. Also, placement of the adhesive around the extremity is sometimes difficult because a single slip will cause permanent improper disposition of the cover. Attempting to reseat the adhesive most frequently tears the plastic and renders the cover unusable. Moreover, I have found the use of hook and loop fasteners enables one to use a single diameter cover for a wide range of leg or arm diameters, thereby eliminating requirements for many different models for differently sized extremities.

Another prior art show the use of adhesive which is attached to an elastic strap to be wound around the opening of the protector and adhered or ‘stuck’ into position. In both cases, if the position is not accurate the first time, the adhesive either would damage the protector during its removal and readjustment or it would lose its ability to stick effectively on a second attempt, rendering it useless for its intended purpose, or after only a single use. Furthermore, should the adhesive contact water or other contaminant prior to bonding with the receiving material, the seal would not take hold in the initial attempt and could never be used as intended.

Additionally, each prior example requires the excess edge of the sleeve covering to rise above the elastic, forming a lip or cupped edge which would collect water during the process of bathing, and lead to considerable seepage during removal of the protector.

One of the more common and inexpensive ways to cover casts and bandages is to use a trash bag and place a rubber band or duct tape around the opening to exclude water. Rubber bands are not overly effective because they frequently do not adequately gather all of the folds of the trash bag, especially since the folds usually randomly encircle the extremity because of the large diameter of the opening relative to the diameter of the extremity being protected. Such random folds provide access for the entry of water. Duct tape is a more effective sealing mechanism but a trash bag must be destroyed each time the duct tape is removed because of the strength of the adhesive.

The current invention involves the use of elastic bands in top and bottom in all size casts from ankle to thigh. The strength and stretch-ability of the bands provide a snug, but comfortable fit for the wearer, preventing water seepage. Located at the open edge of the device, each piece contains elastic band placed parallel and separated by a space, mostly for all items. These bands are attached in such manner that allows the elastic to be positioned in the interior of the finished device. Placed in this manner, these bands allow both single-handed application for the wearer and the flexibility to conform to the movement of the limb.

There is no need for adjustment, since the band relies on its ‘memory’ ability to stretch and return, and there is no need for concern of over-tightening. Additionally, the placement of the elastic prevents water seepage and the double band design allows for added protection if needed, allowing the casing of the inner band to serve as a ledge or ‘catcher’ for seepage.

The size of the opening of the present invention is reasonably larger than an average limb size, but not excessive, such as with a garbage bag, which eliminates the possibility of deep gathers caused by the elastic, thus eliminating the chance of leakage. The elastic is of a content known for its memory, therefore stretching beyond the circumference of the opening, but returning to its original size, which allows the item to be re-used.

The simple design of these items would allowsimilar items to be made in like manner for other parts of the body.

### BRIEF SUMMARY OF THE INVENTION

In the view of the foregoing disadvantages inherent in the known types of cast covers now present in the prior art, the present invention provides an improved disposable and reusable. As such, the general purpose of the present invention, which will be described subsequently in greater detail, is to provide a new and improved disposable and reusable cast cover which has all the advantages of the prior art and none of the disadvantages.

The purpose of the present invention is to eliminate the problems of the prior inventions and create a disposable, yet re-usable and affordable design that will offer protection for the wearer from harmful or damaging fluids, moisture or other contaminants.

This invention will provide a simple, yet effective covering designed for comfort and single-handed application to allow the wearer adequate coverage for normal daily routines, such as bathing, and more independence to perform these activities, making recovery time simpler and easier.

This present invention includes the use of a water-resistant plastic material preferably clear, colored or printed but not limited to color or clarity, and formed into a protective cover for the limbs or other body areas and gathered at the openings with elastic band sealed to the edges, and in some items, with a second band sealed into a cuff in the interior of the device, a distance parallel to the band on the end.

The design of this invention will avoid circulatory problems of over-tightening and the problems of single-use adhesives, ties, straps, Velcro closures or other complicated devices needed to secure a reasonably leak-proof opening, all of which are present in prior inventions.

The simplicity of this present design supports the ability to be affordably disposable, although re-usable, which will promote an environment free of harmful mildew or bacteria for the injured site and the option to discard and replace as needed, should the protector become damaged or contaminated. The prolonged use of a waterproof item can often lead to such problems, even with the most attentive care, which can delay the healing process, or create further health-related issues.

It is another object of the invention to provide a water resistant reusable sleeve for medical dressings to protect a cast for as long as necessary.

Still another object of the invention is to provide a water resistant reusable sleeve for medical dressings which are available in all size.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty which characterize the invention, are pointed out with particularity in the disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 depicts an example of the mitten design for cast cover according to present invention.

FIG. 2 depicts a variation of the arm design for cast covering according to present invention.

FIG. 3 depicts a variation of the foot design for cast covering according to present invention.

FIG. 4 depicts a variation of the leg design for cast covering according to present invention.

### DETAILED DESCRIPTIONS OF THE INVENTION

In the following detailed description, reference is made to the accompanying figures which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description is, therefore, not to be taken in a limiting sense, and the scope of the present invention is defined by the appended description and their equivalents.

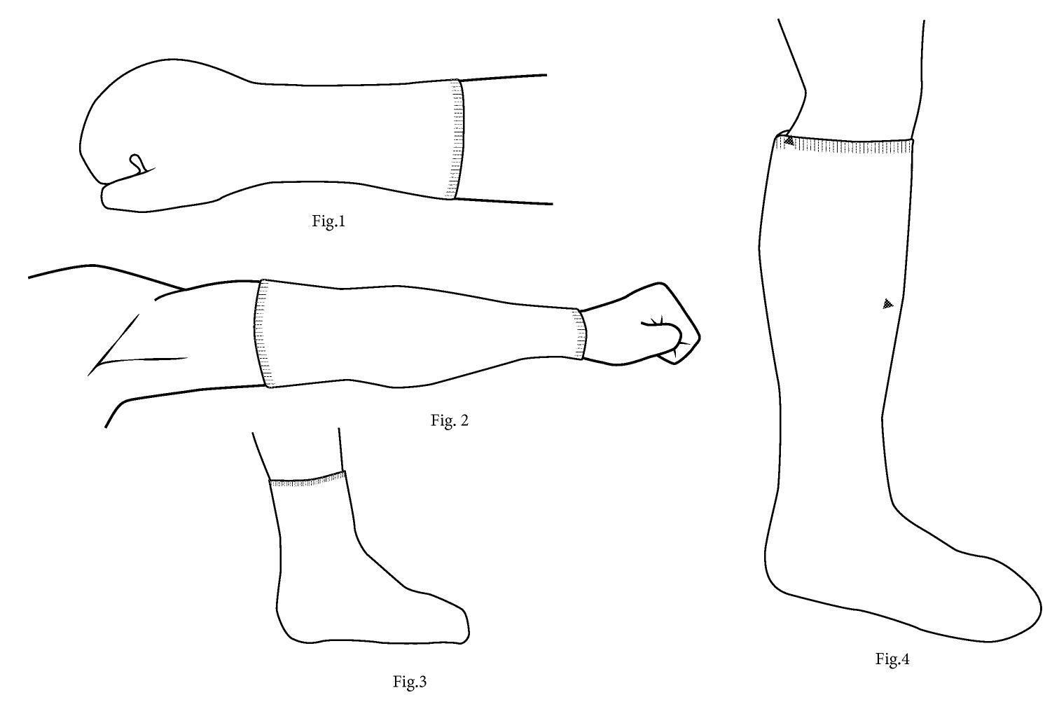

The present invention is directed to a water resistant reusable sleeve for medical dressings, inclusive of medical bandages and casts The preferred embodiment of the invention is depicted in FIGS. 1 and 4.

For the purpose of the specification, the term medical dressing may be understood to comprise casts, bandages, or any other protective material covering or support for an injured or wounded site. Casts and bandages provide a critical part of the therapeutic regimen for broken and wounded limbs. However, medical dressings, especially casts and bandages, quickly lose their usefulness and form when exposed to excessive amounts of moisture, such as may occur during rain, bathing, and showering. Once this occurs, a patient is forced to return to his physician or nurse and have his cast or surgical dressing replaced, which can be an expensive and time-consuming undertaking. Even more dangerous is the fact that the loosening of a cast and the wetting of bandage can result in inadequate healing or infective processes being initiated. Moisture provides an excellent medium for the growth of fungi and bacteria, possibly leading to deleterious results.

In preferred embodiment the present invention provides plastic and elastic disposable and reusable cast covers with elastic on top and bottom in all sizes as shown in FIGS. Preferably different cast designs are shown are available in different sizes.

In one another embodiment FIG. 1 to FIG. 4 shows the illustrative example of the present design for different areas, such as.

Hand wrist: like a mitten with elastic at elbow.

Arm cast: plastic sleeve from wrist to arm pit with elastic on both ends,

Foot cast: like a sock with elastic at knee

Leg cast; plastic sleeve from toe to thigh with elastic at both ends.

It is to be understood that the invention is not limited in its application to the details of construction and to the arrangements of the components set forth in the above description.

As shown in FIGS. 1 to 4, in the preferred embodiment of the invention, the second end portion of the reusable sleeve may have attached thereon a hand portion dimensioned and adapted to receive a hand. In a similar alternative embodiment, such as seen in FIG. 1, a foot portion may be integrally attached to the second end portion. However, the cast cover of foot goes over the foot up to the knee which is dosed in at the toe like a sock so no water can get in while at the time of showering. Moreover, use of either a foot portion or a glove or hand portion extending integrally from or as part of the reusable sleeve may comprise configurations thereof adapted to the specific dimensions of a foot and hand, respectively. For example, individual finger portions may be included, allowing the wearer of a bandage to more fully utilize their hands.

It is to be understood that the above description is intended to be illustrative, and is not restrictive. For example, the above-discussed embodiments may be used in combination with each other. Many other embodiments will be apparent to those of skill in the art upon reviewing the above description.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fail within the scope of the invention.

I claim:

1. A water resistant reusable sleeve for medical dressing comprises of:
a water resistant material having a first end and a second end opposite the first end, and
an elastic band attached into the water resistant material at said ends.

2. The water resistant reusable sleeve for medical dressing of claim 1 available in all sizes for covering Foot cast, Hand cast, Arm cast, and Leg cast.

3. A water resistant reusable sleeve for medical dressing of claim 1, wherein said water resistant material shaped to encircle the first portion.

4. The water resistant reusable sleeve for medical dressing of claim 1, wherein said water resistant material is plastic.

5. The water resistant reusable sleeve for medical dressing of claim 4, wherein said plastic material is clear, colored or printed.

6. The water resistant reusable sleeve for medical dressing of claim 2, wherein said sleeve having a sock shape with said elastic band at knee.

7. The water resistant reusable sleeve for medical dressing of claim 2, wherein said sleeve having a mitten shape with said elastic band at elbow and wrist.

8. The water resistant reusable sleeve for medical dressing of claim 2, wherein said sleeve covering the arm portion of cast from wrist to arm pit with said elastic band at said first end and said second end.

9. The water resistant reusable sleeve for medical dressing of claim 2, wherein said sleeve covering leg cast from toe to thigh with said elastic band at said first end and said second end.

10. The water resistant reusable sleeve for medical dressing of claim 1, is water resistant and disposable.

11. A method of making water resistant reusable sleeve for medical dressing comprising the steps of sewing the elastic band to the first end and a second end opposite the first end of tubular plastic material, such that when in use the elastic band confines the first end of the water resistant material to the snug engagement with the second end of the person's body, said second end opposite to the said first end so as to prevent entry of water inflow.
